(19) United States
(12) Patent Application Publication
(10) Pub. No.: US 2011/0003352 A1
(43) Pub. Date: Jan. 6, 2011

(54) PROCESS FOR THE STEPWISE TREATMENT OF LIGNOCELLULOSIC MATERIAL TO PRODUCE REACTIVE CHEMICAL FEEDSTOCKS

(75) Inventors: Theodora Retsina, Atlanta, GA (US); Vesa Pylkkanen, Atlanta, GA (US); Rolf Ryham, Suwannee, GA (US)

Correspondence Address: Altera Law Group, LLC, 220 S 6 St Suite 1700, Minneapolis, MN, 55402, US

(73) Assignee: AMERICAN PROCESS, INC., Atlanta, GA (US)

(21) Appl. No.: 12/304,046

(22) PCT Filed: Jun. 12, 2007
(86) PCT No.: PCT/US07/13715

§ 371 Date: Sep. 24, 2010

Related U.S. Application Data
(63) Continuation-in-part of application No. 11/740,923, filed on Apr. 27, 2007, now abandoned.
Application No. 11/740,923 is a continuation of application No. 60/795,487, filed on Apr. 28, 2006.
(60) Provisional application No. 60/812,244, filed on Jun. 12, 2006.
(60) Provisional application No. 60/812,245, filed on Jun. 12, 2006.
(60) Provisional application No. 60/812,246, filed on Jun. 12, 2006.
(60) Provisional application No. 60/818,342, filed on Jul. 5, 2006.
(60) Provisional application No. 60/876,470, filed on Dec. 22, 2006.

(51) Int. Cl. C12P 7/40; C08B 5/00; C12P 7/04; C07G 1/00; C13K 1/02; B01D 3/00

(52) U.S. Cl. 435/136; 536/58; 435/157; 530/507; 127/37; 202/176; 203/42

(57) ABSTRACT

A method for the fractionation of lignocellulosic materials into reactive chemical feedstock in a batch or semi continuous process by the stepwise treatment with aqueous aliphatic alcohols in the presence of sulfur dioxide or acid. Lignocellulosic material is fractionated in a fashion that cellulose is removed as pulp, or converted to esterified cellulose, cooking chemicals are reused, lignin is separated in the forms of reactive native lignin and reactive lignosulfonates and hemicelluloses are converted into fermentable sugars, while fermentation inhibitors are removed. In an integrated vapor compression stripper and evaporator system, aliphatic alcohol is removed from a liquid stream and the resulting stream is concentrated for further processing.

### FIELD OF THE INVENTION

This invention relates, in general, to the fractionation of lignocellulosic material into lignin, cellulose and fermentable hemicelluloses, and more particularly to the production of reactive lignin and reactive cellulose in a semi-continuous or batch process. The reactive materials can be used as feedstock for a variety of chemical syntheses including alcohols, organic acids, polymers and other bioproducts.

### BACKGROUND OF THE INVENTION

Fractionation technologies of lignocellulosic material into its main subcomponents of cellulose, lignin and hemicelluloses have existed both in commercial practice and at the research level. The most prevalent of these are commercial sulfite pulping and the U.S. National Renewable Energy Laboratory, NREL, clean fractionation technology research.

Commercial sulfite pulping has been practiced since 1874. The focus of sulfite pulping is the preservation of cellulose which is produced in a crystalline non-reactive form. In an effort to do that, industrial variants of sulfite pulping use a single step process which takes 6-10 hours. In this prolonged processing the hemicelluloses and lignin are dissolved, some hemicelluloses are hydrolyzed into sugars, and then some sugars are converted to organic acids resulting in an overall a low yield of fermentable sugars and converting all lignin to lignosulfonates.

Sulfite pulping produces spent cooking liquor termed sulfite liquor. Fermentation of sulfite liquor to hemicellulosic ethanol has been practiced primarily to reduce the environmental impact of the discharges from sulfite mills since 1909. Published design data from one of the two known remaining sulfite mills that produces ethanol, shows ethanol yields not to exceed 33% of original hemicelluloses. Ethanol yield is low due to the incomplete hydrolysis of the hemicelluloses to fermentable sugars and further compounded by sulfite pulping side products, such as furfural, methanol, acetic acid and others, inhibiting fermentation to ethanol.

Because of poor ethanol yield, lower cost of synthetic ethanol production from oil feed stock, and the production of ethanol from corn today, only two sulfite mills are known to have continued the practice of hemicellulosic ethanol production to date.

In the 20^{th} century, Kraft pulping eclipsed sulfite pulping as the dominant chemical pulping method. Kraft pulping however does not fractionate lignocellulosic material into its primary components in a reactive form. Instead, hemicelluloses are in solution with soluble inorganic cooking chemicals and non-reactive lignin, (condensed lignin with no active site available for chemical bonding), and the lignin cannot readily be separated.

Other processes using solvent cooking chemicals have been tried as an alternative to Kraft or sulfite pulping. The original solvent process is described in U.S. Pat. No. 1,856,567 to Kleinert et al. Although three demonstration size facilities for ethanol-water (ALCELL), alkaline sulfite with anthraquinone and methanol (ASAM), and ethanol-water-sodium hydroxide (Organocell) were operated briefly in the 1990's, today there are no full scale solvent pulping mills. Of these technologies only ALCELL produced native reactive lignin by the use of pure aqueous organic solvents in elevated thermodynamic conditions. None of these technologies produced reactive cellulose or hydrolyzed hemicelluloses.

Groombridge et al. in U.S. Pat. No. 2,060,068 shows that an aqueous solvent with sulfur dioxide is a potent delignifying system to produce cellulose from lignocellulosic material. This process produces non-reactive cellulose for papermaking and the hemicelluloses and lignin are not fractionated.

Furthermore, U.S. Pat. No. 5,879,463 to Proenca reveals that simultaneous delignification and rapid hydrolysis of the entire cellulosic material, both the cellulose and the hemicelluloses, is possible in the presence of an organic solvent and a dilute inorganic acid; however this process does not preserve the cellulose.

Finally, in U.S. Pat. No. 5,730,837 to Black et al. claims fractionation of lignocellulosic material into lignin, cellulose and dissolved sugars using ketone, alcohol, water and mineral acid. This is more readily known as the NREL clean fractionation technology. The lignin so produced is not all in a reactive form because of the use of strong acid that condenses some of the lignin.

In most wood based biorefinery processes a major part of total consumed energy is used to concentrate the extracted sugars and/or organic components to a concentration that is useful for downstream processing. In the Kraft process this is done with multiple effect evaporators which use steam. In corn ethanol processes the distillation of ethanol and evaporation of water from the distillers grain is done in a two step process using steam. In U.S. Pat. No. 6,217,711 to Ryham et al, the stripping of methanol and the concentration of black liquor was achieved in an integrated process in which the liquor was first concentrated and the methanol was then stripped from it. In the present invention an integrated stripper and evaporator are designed to first remove aliphatic alcohol from a stream and then to concentrate the stream.

Therefore in the prior art of processing lignocellulosic material:

a) The sulfite processes to date preserves cellulose in a non-reactive form, degrades some hemicelluloses into non-reactive byproducts and sulfonates all lignin.
b) The Kraft process does not fractionate lignin, cellulose and hemicelluloses
c) Organic solvent pulping methods produced a non-reactive cellulose and did not hydrolyze hemicelluloses. Furthermore they used pressures and temperatures significantly higher than conventional pulping methods.
d) Treatment of lignocellulosic material with dilute inorganic acid in organic solvent hydrolyzes both cellulose and hemicelluloses and therefore does not preserve cellulose
e) Treatment of lignocellulosic material with ketone, alcohol, water and mineral acid does not produce all lignin in a reactive form.

The present inventors have now developed a process for the treatment of lignocellulosic material which first fractionates the material and then converts each fraction into a reactive chemical feedstock. This is achieved through cooking lignocellulosic material with sulfur dioxide in a solution of ethanol and water in a one or multiple stage process where treatment of each fraction is continued after intermediary separation of the fractions and such treatment conditions are modified to achieve desired fraction final properties. This can be done in a batch or semi continuous process.

Surprisingly, such a process can isolate at least four reactive components within one multiple stage integrated process treatment and provide four industrial feedstock chemicals in large scale.

These are:

1) cellulose which can be diverted to paper making or be further treated to produce reactive esterified cellulose suitable for industrial production of synthetic fibers and polymers, or for high yield ethanol production. In the esterification, cellulose is converted into an amorphous form where the free hydroxyl groups are exposed to further reaction.
2) fermentable hemicelluloses and fermentable sugars suitable for high yield ethanol production
3) reactive native lignin, i.e., lignin dissolved by alcoholysis and is in near original polymer length and its reactive sites are preserved in the solution
4) reactive lignosulfonates, i.e., lignin that has been partially sulfonated but retains near original polymer length and can further react with electrolytes.

Molecular sieves are the common method of breaking the water-ethanol azeotrope in corn ethanol plants. Molecular sieves consume at least 150 kilojoules of energy per liter of ethanol to remove the last 4.5% of water, which is more energy than traditional distillation of water. Use of lime for dehydrating ethanol is common practice in laboratories but an industrial scale application is cost prohibitive because of the cost of regenerating lime from its resultant hydrated state to the required anhydrous state, and the associated loss of ethanol captured in the hydrated lime. Coincidentally, it was realized in the present invention that the hydrated lime byproduct can be reintroduced to the process in the lignin precipitation step. Therefore, the lime is beneficially used in the process and the ethanol captured in the hydrated lime is recovered to the process.

### BRIEF SUMMARY OF THE INVENTION

On aspect of the invention is a process for fractionating lignocellulosic material in to chemically reactive components through a staged treatment of the lignocellulosic material with a solution of aliphatic alcohol, water and sulfur dioxide with intermediate separation of cellulose and hemicelluloses-lignin fractions which are then each further treated with a solution of aliphatic alcohol, water and sulfur dioxide or acid.

Another aspect is a process wherein said solution of aliphatic alcohol, water and sulfur dioxide contains 40% to 60% water.

Another aspect is a process wherein a different concentration of said solution of aliphatic alcohol, water and sulfur dioxide is used at a first stage of treatment of said lignocellulosic material than is used in one or more subsequent stages of treatment with intermediate removal and preservation of cellulose.

Another aspect is a process wherein a sulfur dioxide solution of 3% to 20% is used at a first stage of treatment and a sulfur dioxide, sulfurous acid or sulfuric acid solution of 0.5% to 20% is used in one or more subsequent stages of treatment with intermediate removal and preservation of cellulose.

Another aspect is a process wherein said process is followed by steam stripping and/or evaporation of hydrolyzate to remove and recover sulfur dioxide and aliphatic alcohol and to remove fermentation inhibitors.

Another aspect is a process wherein cellulose is further treated with an aqueous solution of aliphatic alcohol in the presence of acid to esterify and render it a reactive chemical feedstock.

Another aspect is a process wherein the esterified cellulose is fermented to aliphatic alcohol.

Another aspect is a process wherein said acid is sulfur dioxide, sulfurous acid or sulfuric acid solution of 0.5% to 20%.

Another aspect is a process wherein preferred conditions are 95% ethanol and 5% sulfuric acid at 120° C.

Another aspect is a process wherein said process is carried out at temperatures between 65° C. and 200° C.

Another aspect is a process wherein said process is carried out at for a period of time between 15 minutes and 720 minutes.

Another aspect is a process wherein preferred conditions are an initial treatment using 47% ethanol, 47% water and 6% sulfur dioxide at 140° C. for 2 hours, and following the intermediate removal and preservation of the cellulose, a further treatment of the hemicelluloses-lignin fraction using 2% ethanol, 95% water and 3% sulfur dioxide at 140° C. for 1 hour.

Another aspect is a process is producing fermentable sugars from the hemicelluloses of a lignocellulosic material through a staged treatment of the lignocellulosic material with a solution of aliphatic alcohol, water and sulfur dioxide with intermediate removal of hydrolyzate and cellulose.

Another aspect is a process wherein a different concentration of said solution of aliphatic alcohol, water and sulfur dioxide is used at a first stage of treatment of said lignocellulosic material than is used in one or more subsequent stages of treatment with intermediate removal of hydrolyzate and cellulose.

Another aspect is a process wherein said process is carried out at for a period of time between 15 minutes and 720 minutes.

Another aspect is a process wherein aliphatic alcohol is produced from fermenting and distilling hydrolyzed fermentable sugars produced in said process and is then reused in said process.

Another aspect is a process wherein lignin is sulfonated and rendered soluble in aqueous solutions.

Another aspect is a process wherein the concentration of sulfur dioxide and aliphatic alcohol in the solution and the time of cook is varied to control the yield of hemicelluloses vs. celluloses and vs. fermentable sugars.

Another aspect is a process wherein excess sulfur dioxide is released from said further treatment of each fraction and used for make-up for cooking chemicals.

Another aspect is a process for fractionating lignocellulosic material in to chemically reactive components through a staged treatment of the lignocellulosic material with a solution of aliphatic alcohol, water and sulfur dioxide with intermediate separation of cellulose and hemicelluloses-lignin fractions which are then each further treated with a solution of aliphatic alcohol, water and sulfur dioxide or acid. comprising the steps of:

Cooking under acidic conditions to produce hydrolyzed hemicelluloses, cellulose, reactive lignin and sulfonated lignin;
Washing to separate lignin and hemicelluloses from cellulose in several stages to recover over 95% of the aliphatic alcohol mixed with the cellulose;
Diverting the cellulose to papermaking or treatment of cellulose with an aqueous solution of aliphatic alcohol in the presence of acid to esterify the cellulose, rendering it reactive and thereby a suitable chemical feedstock
Treatment of post washing hydrolyzate with sulfur dioxide and heat to maximize the yield of fermentable sugars and to remove, and/or neutralize fermentation inhibitors;
Evaporation to remove and recover cooking chemicals, remove side products, precipitate reactive native lignin and concentrate lignosulfonates and/or fermentable sugars product;
Lignin separation to remove reactive native lignin and reactive lignosulfonates from fermentable sugars;
Fermentation and distillation to produce and concentrate aliphatic alcohols or organic acids;
Drying the concentrated aliphatic alcohols or organic acids with anhydrous lime and reusing the resulting hydrated lime byproduct for lignin separation; and
Fractionation and/or separation to remove and recover side products.

Another aspect is a process further comprising the step of fractionation and/or separation to remove and recover side products.

Another aspect is a process further comprising the step of lignin and/or lignosulfonate separation.

Another aspect is a process wherein aliphatic alcohol soluble lignin is separated by evaporation of said aliphatic alcohol and subsequent removal of reactive native lignin precipitate.

Another aspect is a process wherein reactive lignosulfonates are selectively precipitated using excess lime in the presence of aliphatic alcohol.

Another aspect is a process wherein lignosulfonates filter cake is combusted in a fluidized bed boiler or gasifier, sulfur is released and reacts with excess lime in said filter cake to form gypsum.

Another aspect is a process further comprising the step of fermentation and distillation.

Another aspect is a process wherein anhydrous lime is used to dry product aliphatic alcohol from distillation and resultant hydrated lime byproduct is reused to displace fresh lime in the associated upstream feedstock preparation process.

Another aspect is a process wherein said hydrated lime byproduct is used in claim 26 to precipitate lignosulfonates.

Another aspect is a process wherein aliphatic alcohol is removed from a stream and the resulting stream is concentrated in an integrated alcohol stripper and evaporator system, wherein the evaporated vapor is compressed using vapor compression and provides the thermal energy for both the stripper and the evaporator.

Another aspect is a process wherein evaporated vapor streams are segregated so as to have different concentrations of organic compounds in different streams.

Another aspect is a process wherein evaporator condensate streams are segregated so as to have different concentrations of organic compounds in different streams.

Another aspect is a process wherein the evaporator condensates and distillation column bottoms are used to wash cellulose to minimize effluent discharges.

Further, the present invention describes a process of fractionating lignocellulosic material into lignin, cellulose and hydrolyzed hemicelluloses through a staged treatment of the lignocellulosic material with a solution of aliphatic alcohol, water and sulfur dioxide, in multiple step process where:
the cellulose is first fractionated and in an intermediary step and can then diverted to further treatment,
the hemicellulose and sugar rich stream, which also contains the cooking chemicals and particularly the aliphatic alcohol, is treated to remove and recycle the aliphatic alcohol and to remove water to concentrate the stream so it is suitable for downstream processing
the hemicelluloses are converted to fermentable sugars, and fermentation inhibitors are removed
the native reactive lignin is separated from the hemicelluloses
the reactive lignosulfonates are separated.

Hence in a preferred embodiment lignocellulosic material is treated in a first stage with aliphatic alcohol, water and sulfur dioxide, the cellulose is then removed, and then both fractions are each further treated with aliphatic alcohol, water, sulfur dioxide or acid. Aliphatic alcohol is stripped and removed from the resulting liquid stream and then the stream is concentrated in an integrated alcohol stripper and evaporator system, wherein the evaporated vapor is compressed using vapor compression and provides the thermal energy for both the stripper and the evaporator. Alcohol soluble lignin is separated from the precipitate or sulfonated by adding sulfur dioxide and heat. Addition of hydrated lime will precipitate alkali insoluble lignosulfonates. Remaining sugar solution is fermented and distilled of ethanol. Distillation column bottoms and condensate are used for pulp washing. Finally the product ethanol is refluxed over lime to remove water from azeotropic solution.

It is noted that the summary is merely a guide to the contents of the entire application which must be read to understand the claims which define the invention.

### BRIEF DESCRIPTION OF THE DRAWINGS

A more complete understanding of the present invention may be obtained by reference to the following detailed description when read in conjunction with the accompanying drawings wherein:

FIG. 1. Illustrates the products obtained from the fractionation of lignocellulosic material;

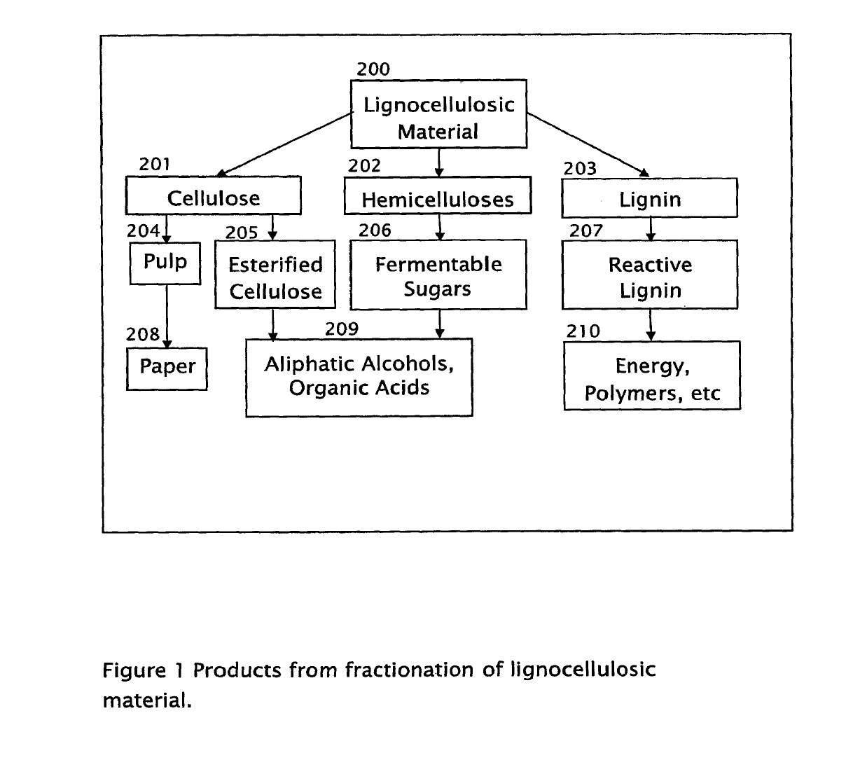

FIG. 2. Illustrates a flow sheet example of the invention process, noting that the process steps may be in other sequences and

FIG. 3. Illustrates a flow sheet example of the invention's integrated alcohol stripper and evaporator system.

### DETAILED DESCRIPTION OF THE INVENTION

A method and system for the fractionation of lignocellulosic materials into reactive chemical feedstock in a batch or semi continuous process by the stepwise treatment with aqueous aliphatic alcohols in the presence of sulfur dioxide or acid. Lignocellulosic material is fractionated in a fashion that cellulose is removed as pulp, or converted to esterified cellulose, cooking chemicals are reused, lignin is separated in the forms of reactive native lignin and reactive lignosulfonates and hemicelluloses are converted into fermentable sugars, while fermentation inhibitors are removed. In an integrated vapor compression stripper and evaporator system, aliphatic alcohol is removed from a liquid stream and the resulting stream is concentrated for further processing.

A process for fractionating lignocellulosic material in to chemically reactive components through a staged treatment of the lignocellulosic material with a solution of aliphatic alcohol, water and sulfur dioxide with intermediate separation of cellulose and hemicelluloses-lignin fractions which are then each further treated with a solution of aliphatic alcohol, water and sulfur dioxide or acid, comprising the steps of:

Cooking under acidic conditions to produce hydrolyzed hemicelluloses, cellulose, reactive lignin and sulfonated lignin, wherein the concentration of sulfur dioxide and aliphatic alcohol in the solution and the time of cook is varied to control the yield of hemicelluloses vs. celluloses and vs. fermentable sugars;

Washing to separate lignin and hemicelluloses from cellulose in several stages to recover over 95% of the aliphatic alcohol mixed with the cellulose;

Diverting the cellulose to papermaking or treatment of cellulose with an aqueous solution of aliphatic alcohol in the presence of acid to esterify the cellulose, rendering it reactive and thereby a suitable chemical feedstock
Treatment of post washing hydrolyzate with sulfur dioxide and heat to maximize the yield of fermentable sugars and to remove, and/or neutralize fermentation inhibitors;
Evaporation to remove and recover cooking chemicals, remove side products, precipitate reactive native lignin and concentrate lignosulfonates and/or fermentable sugars product;

Lignin separation to remove reactive native lignin and reactive lignosulfonates from fermentable sugars;

Fermentation and distillation to produce and concentrate aliphatic alcohols or organic acids;
Drying the concentrated aliphatic alcohols or organic acids with anhydrous lime and reusing the resulting hydrated lime byproduct for lignin separation; and
Fractionation and/or separation to remove and recover side products.

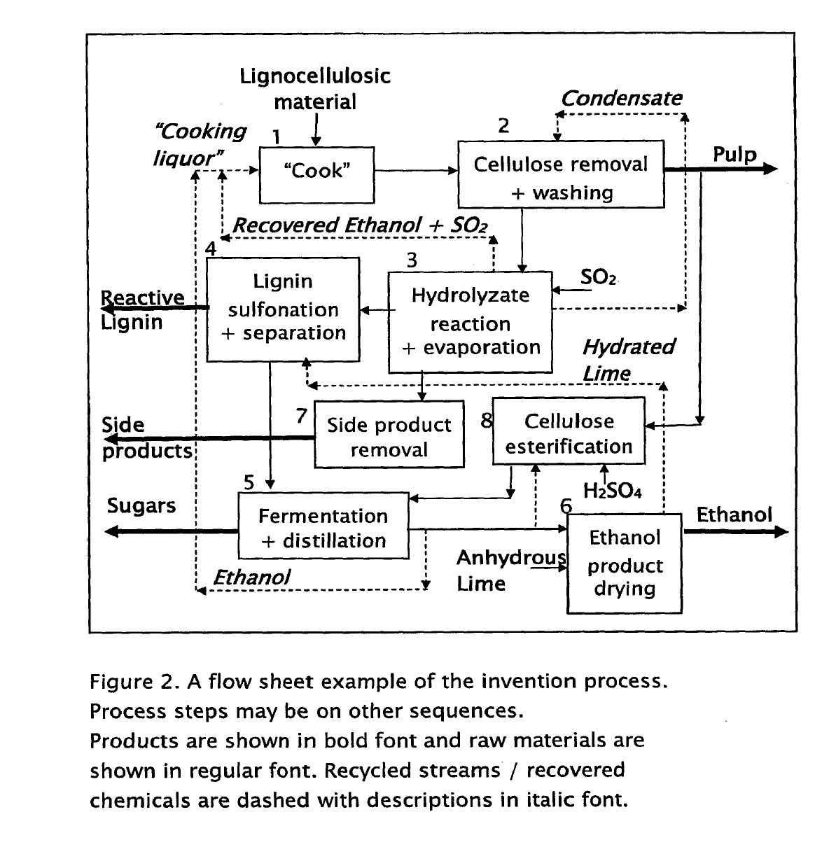

The first process step is “cooking”, element 1 in FIG. 2, which fractionates the lignocellulosic material components to allow easy downstream removal; specifically hemicelluloses are dissolved and over 50% are completely hydrolyzed, cellulose is separated but remains resistant to hydrolysis, and lignin is sulfonated in water soluble form. Lignoceliulosic material is processed, “cooked”, in a solution of aliphatic alcohol, water, and sulfur dioxide where typical ratios by weight are 40-60% of both aliphatic alcohol and water, and 0.5-20% of sulfur dioxide, and preferably 47% aliphatic alcohol, 47% water, and 0.5-20% sulfur dioxide; this solution is termed cooking liquor. Aliphatic alcohols can include ethanol, methanol, propanol and butanol, but preferably ethanol. The cooking is performed in one or more stages using batch or continuous digesters. Depending on the lignocellulosic material to be processed, the cooking conditions are varied, with temperatures from 65° C. to 200° C., for example 65° C., 75° C., 85° C., 95° C., 105° C., 115° C., 125° C., 130° C. 135° C., 140° C. 145° C., 150, ° C., 155° C., 165° C., 170, ° C., 180° C., 190° C. or 200° C., and corresponding pressures from 1 atmosphere to 15 atmospheres. The sulfur dioxide charge in the cooking liquor is varied between 0.5% and 20%, for example 0. 5%, 1%, 1.5%, 2%, 2.5%, 3%, 3.5%, 4%, 5%, 6%, 7%, 8%, 9%, 10%, 11%, 12%, 13%, 14%, 15%, 16%, 17%, 18%, 19% or 20% of the total cooking liquor mass in one or more cooking stages. Cooking time of each stage is also varied between 15 minutes and 720 minutes, for example 15, 30, 45, 60, 90, 120, 140, 160, 180, 210, 240, 270, 300, 330, 360, 390, 420, 450, 480, 510, 540, 570, 600, 630, 660, 690 or 720 minutes. The lignocellulosic material to cooking liquor ratio can is varied between 1:3 to 1:6, for example, 1:3, 1:4, 1:5 or 1:6, and preferably 1:4.

Hydrolyzate from the cooking step is subjected to pressure reduction, either at the end of a cook in a batch digester, or in an external flash tank after extraction from a continuous digester. The flash vapor from the pressure reduction is collected into a cooking liquor make-up vessel. The flash vapor contains substantially all the unreacted sulfur dioxide which is directly dissolved into new cooking liquor. The cellulose is then removed to be washed and further treated as required.

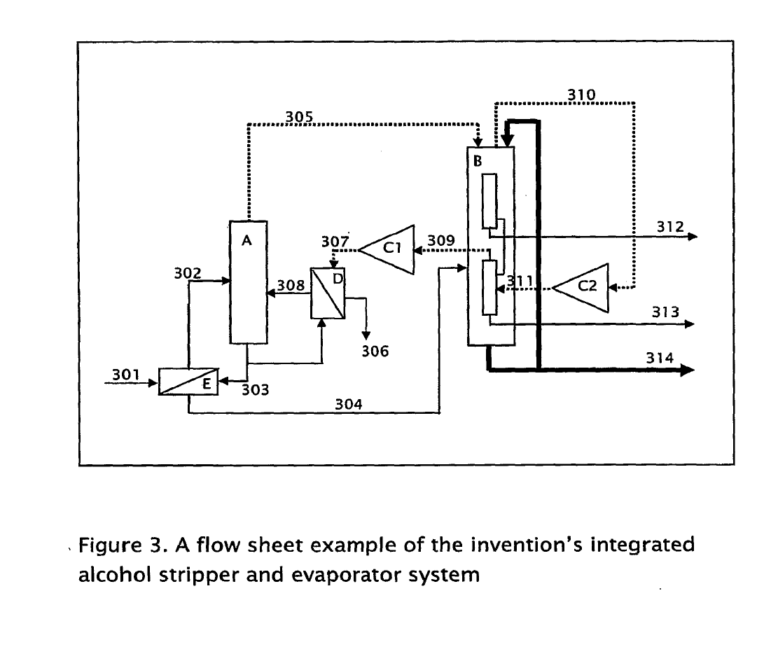

The process washing step, element 2 in FIG. 2, recovers the hydrolyzate from the cellulose. The washed cellulose is pulp that can be used for paper production or esterification. The weak hydrolyzate from the washing step continues to the post washing hydrolyzate reaction step, element 3 in FIG. 3; in a continuous digester application this weak hydrolyzate will be combined with the extracted hydrolyzate from the external flash tank.

In the post washing hydrolyzate reaction step, the post washing hydrolyzate is further treated in one or multiple steps with a solution of aliphatic alcohol, water, and sulfur dioxide, sulfurous acid or sulfuric acid, where typical ratios by weight of aliphatic alcohol to water is between 1:99 and 50:50, for example 1:99, 2:98, 3:97, 4:96, 5:95, 10:90, 20:80, 30:70, 40:60, and 50:50, and sulfur dioxide, sulfurous acid or sulfuric acid to a charge of 0.5% and 20%, for example 0. 5%, 1%, 1.5%, 2%, 2.5%, 3%, 3.5%, 4%, 5%, 6%, 7%, 8%, 9%, 10%, 11%, 12%, 13%, 14%, 15%, 16%, 17%, 18%, 19% or 20%, and directly or indirectly heated to temperatures up to 200° C., for example 105° C., 115° C., 125° C., 135° C., 140° C., 145° C., 150° C., 155° C., 160° C. 170° C., 180° C. 190° C. or 200° C., and preferably 140° C. Said solution may or may not contain residual alcohol. This step produces fermentable sugars which can then be concentrated by evaporation to a fermentation feedstock. Concentration by evaporation can be before or after the treatment with sulfur dioxide, sulfurous or sulfuric acid in said post washing hydrolyzate reaction step. This step may or may not be followed by steam stripping of the resultant hydrolyzate to remove and recover sulfur dioxide and aliphatic alcohol and for removal of potential fermentation inhibiting side products. The evaporation process may be under vacuum or pressure from −0.1 atmospheres to 3.0 atmospheres, for example 0.1, 0.3, 0.5, 1.0, 1.5, 2.0, 2.5, or 3.0 atmospheres. Aliphatic alcohol is recovered from the evaporation process by condensing the exhaust vapor and is returned to the cooking liquor make-up vessel in the cooking step. Clean condensate from the evaporation process is used in the washing step. The hydrolyzate from the evaporation and post washing hydrolyzate reaction step contains mainly fermentable sugars but may also contain lignin depending on the location of the lignin separation step in the overall process configuration, and is concentrated between 10% and 55% solids, for example 10%, 15%, 20%, 25%, 30%, 35%, 40%, 45%, 50% or 55%; this hydrolyzate continues to a subsequent process step. In a preferred embodiment the evaporation step comprises the present invention's integrated alcohol stripper and evaporator.

Fermentable sugars are defined as hydrolysis products of cellulose, galactoglucomannan, glucomannan, arabinogalactan, arabinoglucuronoxylans, and glucuronoxylans in to their respective short-chained oligomer and monomer products, i.e., glucose, mannose, galactose, xylose, and arabinose, which are substantially free of fermentation inhibitors. In a preferred embodiment, this is a solution of monomer sugars essentially free of fermentation inhibitors. In a most preferred embodiment it is a solution of monomer sugars with concentration of furfural below 0.15% of the sugars.

Cellulose removed in the washing step can be diverted for papermaking or in a preferred embodiment can be esterified into reactive cellulose by further treatment with aliphatic alcohol in the presence of sulfur dioxide or acid, element 8 in FIG. 2. Aliphatic alcohol will be at a concentration of 96% or higher in the presence of sulfur dioxide, sulfurous acid or sulfuric acid to a charge of 0.5% and 20%, for example 0.5%, 1%, 1.5%, 2%, 2.5%, 3%, 3.5%, 4%, 5%, 6%, 7%, 8%, 9%, 10%, 11%, 12%, 13%, 14%, 15%, 16%, 17%, 18%, 19% or 20%, and directly or indirectly heated to temperatures up to 200° C., for example 100° C., 105° C., 110° C., 115° C., 120° C., 125° C., 130° C., 140° C., 150° C., 160° C. 170° C., 180° C., 190° C. or 200° C., and preferably 120° C. The reactive cellulose can either be sold for use as reactive chemical feed stock, or be fermented into an aliphatic alcohol or organic acid.

The process lignin separation step, element 4 in FIG. 2, is for the separation of lignin from the hydrolyzate and can be located before or after the post washing hydrolyzate reaction step and evaporation. If located after, then reactive native lignin precipitates from the hydrolyzate since aliphatic alcohol has been removed in the evaporation step. The remaining water soluble lignosulfonates are precipitated by converting the hydrolyzate to an alkaline condition using an alkaline earth oxide, preferably fresh lime or, in a preferred embodiment, hydrated lime from the product aliphatic alcohol drying step. The alkaline condition is due to presence of unreacted lime, termed excess lime. The combined lignin and lignosulfonate precipitate is filtered. The lignin and lignosulfonate filter cake can be dried as a saleable byproduct or be burned in a fluidized bed boiler or gasifier for energy production; sulfur released by combustion reacts with excess lime in the filter cake to form gypsum which can be collected and sold as a side product. The hydrolyzate from filtering can either be sold as a concentrated sugar solution product or be further processed in a subsequent fermentation step to aliphatic alcohol.

The process fermentation and distillation step, element 5 in FIG. 2, is for the production of aliphatic alcohols, most preferably ethanol, or organic acids. After removal of cooking chemicals and lignin, and treatment in the post washing hydrolyzate reaction step, the hydrolyzate contains mainly fermentable sugars in water solution from which any fermentation inhibitors have been removed or neutralized. The hydrolyzate is fermented to produce dilute alcohol or organic acids, from 1% to 10% concentration. Spent yeast is removed by filtration. The dilute alcohol is distilled to concentrate to near to its azeotropic point of 95.6% by weight. Some of the alcohol produced from this stage is used for the cooking liquor makeup in the process cooking step. The majority of the alcohol produced is excess and is purified for saleable grade product in the product ethanol drying step. In a preferred embodiment the distillate column bottoms solution and evaporator condensates are used to wash cellulose in the process washing step to minimize effluent discharges.

The aliphatic alcohol product drying step, element 6 in FIG. 2, is for the removal of the water from aliphatic alcohol-water azeotrope. After distillation of the aliphatic alcohol, the remaining water is removed by anhydrous lime, where product aliphatic alcohol vapor is released through a vertical absorption column containing anhydrous lime. Hydrated lime is withdrawn from the bottom of the column in a batch or continuous manner, as a byproduct and can be reused to displace fresh lime in the associated upstream feedstock preparation process. In a preferred embodiment the hydrated lime will be used for precipitation in the lignin separation step, element 4 in FIG. 2. The product aliphatic alcohol is purified to fuel grade alcohol by use of molecular sieves.

The process side products removal step, element 7 in FIG. 2, uses fractionation or separation techniques to remove side products from the hydrolyzate that are of economic value or accumulate to inhibit the yield and quality of the alcohol or pulp products. These side products are isolated by processing the vent from the final reaction step and the condensate from the evaporation step. Side products include furfural, methanol, and acetic acid.

The present invention includes a system for removing aliphatic alcohol from a stream and concentrating the resulting stream comprising an integrated alcohol stripper and evaporator system, as illustrated in the flow sheet provided in FIG. 3, wherein aliphatic alcohol is removed by vapor stripping, the resulting stripper product stream is concentrated by evaporating water from the stream, evaporated vapor is compressed using vapor compression and is reused to provide thermal energy, for both the stripper and the evaporator.

In FIG. 3 the following reference numerals refer to the indicated elements:

| Ref. No. | Elements |
|---|---|
| 301. | Stripper feed. |
| 302. | Preheated stripper feed. |
| 303. | Stripper column bottoms. |
| 304. | Cooled stripper column bottoms. |
| 305. | Stripper column overhead. |
| 306. | Foul condensate. |
| 307. | Reboiler steam supply. |
| 308. | Reboiler to stripper column, or direct steam supply. |
| 309. | Evaporator vapor to stripper column vapor compressor. |
| 310. | Evaporator vapor to evaporator vapor compressor. |
| 311. | Evaporator steam supply. |
| 312. | Aliphatic alcohol. |
| 313. | Evaporator condensate; up to 4 condensate streams of |
| | varying levels of contamination. |
| 314. | Concentrated organic stream. |
| A. | Stripper column. |
| B. | Evaporator. |
| C1. | Stripper column vapor compressor. |
| C2. | Evaporator vapor compressor. |
| D. | Reboiler. |
| E. | Stripper feed heat exchanger. |

The stripper feed (301) is any stream containing aliphatic alcohol for which it is desired to remove and capture the aliphatic alcohol and to concentrate the remaining organic stream for further processing. In one preferred embodiment this will be the weak hydrolyzate from the current invention's process washing step; in a continuous digester application this weak hydrolyzate will be combined with the extracted hydrolyzate from the external flash tank to form the stripper feed. In another preferred embodiment this will be the feed to the beer column in a corn ethanol plant.

The stripper feed is dilute with a concentration between 0.5 and 50% in non water components by weight, for example 0. 5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, and 50% of which aliphatic alcohol is present in concentration between 0.1% and 25% for example 0. 1%, 0.5%, 1%, 2%, 3%, 4%, 5%, 6%, 7% 8%, 9%, 10%, 11%, 12%, 13%, 14%, 15%, 16%, 17%, 18%, 19%, 20%, 21%, 22%, 23%, 24%, and 25%.

The stripper feed is first preheated with the stripper column bottoms (303) in the stripper feed heat exchanger (E) and then directed to the stripper column (A). The stripper column (A) can be operated with a reboiler (D) or by direct steam injection. The stripper column overhead (305) is directed to the evaporator (B) which also acts as a reflux condenser where the stripper column overhead is condensed. The stripper column bottoms (304), after being cooled by heat exchanging with the stripper feed, is directed to the evaporator (B), where it is evaporated. The resulting evaporated vapor is removed from the evaporator and separated into two vapor streams (309) and (310); one (309) is compressed in the stripper column vapor compressor (C1) and directed to the stripper reboiler to provide the required thermal energy for the stripper column, while the other (310) is compressed in the evaporator vapor compressor (C2) and directed to the evaporator to provide the required steam supply.

From one to four different streams of condensate are segregated so as to have different concentrations of organic compounds in different streams, and are removed from the evaporator (313). In a preferred embodiment, one of these streams, usually the cleanest (i.e. the one with the highest water concentration), is used in the process washing step, or in another step in the process where a clean condensate stream is required. The other condensate streams are richer in organic compounds, for example, acetic acid, methanol, furfural, aldonic acids and others, and are directed to the fractionation step. The foul condensate (306) from the reboiler can also be directed to the process fractionation step.

The operating conditions of the stripper column are such that the rejected heat from the evaporator is of high enough temperature to drive evaporation. The present invention's innovation lies in the integration and order of the operations where first the aliphatic alcohol is removed and second the resulting stream is concentrated, while both operations are driven by steam produced from compressing the evaporated vapor to two different operating pressures. As such the stripper must be operated at higher pressure than the evaporator and specifically at pressures between 0.34 and 12 atmospheres for example at 0.34, 0.40, 0.48, 0.54, 0.61, 0.68, 0.75, 0.82, 0.88, 0.95, 1.02, 1.09, 1.16, 1.22, 1.29, 1.36, 1.43, 1.50, 1.57, 1.63, 1.70, 1.77, 1.84, 1.91, 1.97, 2.04, 2.11, 2.18, 2.25, 2.31, 2.38, 2.45, 2.52, 2.59, 2.65, 2.72, 2.79, 2.86, 2.93, 2.99, 3.06, 3.13, 3.20, 3.27, 3.33, 3.40, 3.47, 3.54, 3.61, 3.67, 3.74, 3.81, 3.88, 3.95, 4.01, 4.08, 4.15, 4.22, 4.29, 4.35, 4.42, 4.49, 4.56, 4.63, 4.70, 4.76, 5.10, 5.44, 5.78, 6.12, 6.46, 6.80, 7.14, 7.49, 7.83, 8.17, 8.51, 8.85, 9.19, 9.53, 9.87, 10.21, 10.55, 10.89, 11.23, 11.57, 11.91 atmospheres while the evaporator operates between −1.00 to 16.80 atmospheres, for example −1.00, −0.95, −0.92, −0.88, −0.85, −0.82, −0.78, −0.75, −0.71, −0.68, −0.65, −0.61, −0.58, −0.54, −0.51, −0.48, −0.44, −0.41, −0.37, −0.34, −0.31, −0.27, −0.24, −0.20, −0.17, −0.14, −0.10, −0.07, −0.03, 0, 0.03, 0.07, 0.10, 0.14, 0.17, 0.20, 0.24, 0.27, 0.31, 0.34, 0.37, 0.41, 0.44, 0.48, 0.51, 0.54, 0.58, 0.61, 0.65, 0.68, 0.71, 0.75, 0.78, 0.82, 0.85, 0.88, 0.92, 0.95, 0.99, 1.02, 1.05, 1.09, 1.12, 1.16, 1.19, 1.22, 1.26, 1.29, 1.33, 1.36, 1.39, 1.43, 1.46, 1.50, 1.57, 1.63, 1.70, 1.77, 1.84, 1.91, 1.97, 2.04, 2.11, 2.18, 2.25, 2.31, 2.38, 2.45, 2.52, 2.59, 2.65, 2.72, 2.79, 2.86, 2.93, 2.99, 3.06, 3.13, 3.20, 3.27, 3.33, 3.40, 3.47, 3.54, 3.61, 3.67, 3.74, 3.81, 3.88, 3.95, 4.01, 4.08, 4.15, 4.22, 4.29, 4.35, 4.42, 4.49, 4.56, 4.63, 4.70, 4.76, 5.10, 5.44, 5.78, 6.12, 6.46, and 6.80 atmospheres.

Electrical power is used to drive the vapor compressors (C1) and (C2) to compress the evaporator vapor to the conditions needed to operate the stripper column and the evaporator. Depending on the temperature of the stripper feed and the operating pressures of the stripper and the evaporator the operations may have a shortfall or an excess of heat. In the former case a live steam supply raised in a boiler outside the present invention's process is required to supplement the compressed evaporator vapor streams. In another embodiment, a reboiler is included to raise low pressure clean steam, i.e., less than 4.42 atmospheres, from condensing the stripper column overhead fraction. The clean steam is then compressed to run the evaporator. In this case the reboiler condensate will be combined with any of the evaporator condensates to the process fractionation step or be directed to the process fractionation step on its own.

In a preferred embodiment the evaporator tubes (the space in the evaporator where the vapor is released) is separated into two or more spaces, so that the evaporated vapor streams can be segregated and the concentration of the organic compounds in each released vapor stream can be varied. In this case a selection of the more contaminated vapor may be preferably directed to the stripper reboiler.

Although other modifications and changes may be suggested by those skilled in the art, it is the intention of the inventors to embody within the patent warranted hereon all changes and modifications as reasonably and properly come within the scope of their contribution to the art.

### Example 1

One representative example of a design heat and material balance of the alcohol stripper and evaporator system is given in the following table:

| | | | | | | | Conden- | | | | | | Regenerated | Conden- | Sugar |
|---|---|---|---|---|---|---|---|---|---|---|---|---|---|---|---|
| | | | | | | | sate | | | | | | Alcohol | sate | Product |
| Refer To | | Stream | | | | | | | | | | | | | |
| FIG. 3 | No. | 301 | 302 | 303 | 304 | 305 | 306 | 307 | 308 | 309 | 310 | 311 | 312 | 313 | 314 |
| H₂O | kg/h | 392 | 389 | 380 | 380 | 12 | 125 | 125 | | 125 | 81 | 81 | 12 | 79 | 176 |
| EtO | kg/h | 50 | 50 | 3 | 3 | 48 | 0.4 | 0.4 | | 0.4 | 0.3 | 0.3 | 48 | 1.9 | 0.2 |
| Organics | kg/h | 44 | 47 | 44 | 44 | 0 | 0.05 | 0.05 | | 0.05 | 0.2 | 0.2 | −0.5 | 0.4 | 44 |
| Total | kg/h | 486 | 486 | 427 | 427 | 59 | 126 | 126 | | 126 | 82 | 82 | 59 | 81 | 220 |
| Temp. | ° C. | 97 | 119 | 144 | 106 | 48 | 146 | 177 | | 101 | 101 | 121 | 119 | 101 | 100 |
| Sat. Temp. | ° C. | | | | | 106 | | 146 | | | | 121 | | | |
| Pressure | atm | | | | | | | 4.42 | | 0 | 0 | 0.34 | | | |
| Et conc. | % | 10.3 | 10.3 | 0.6 | 0.6 | 80.2 | 0.3 | 0.3 | | 0.3 | 0.3 | 0.3 | 80.8 | 2.3 | 0.1 |
| “Solids” | % | 19.3 | 20.0 | 10.9 | 10.9 | 80.2 | 0.4 | 0.4 | | 0.4 | 0.6 | 0.6 | 80.0 | 2.8 | 20.2 |
| “Sugars” | % | 9.0 | 9.7 | 10.4 | 10.4 | 0.0 | 0.0 | 0.0 | | 0.0 | 0.2 | 0.2 | −0.8 | 0.4 | 20.1 |

### Example 2

The following example illustrates the invention for producing ethanol from hemicelluloses, but in no way limits it:

Wood chips of mixed northern pine species, containing 42.68% moisture, were cooked for 180 minutes at 157° C. in a 1 liter Parr reactor. The moisture adjusted cooking liquor consisted of 3% SO2, 48.5% of ethanol and 48.5% water by weight in 6 parts of total liquor to 1 part of dry wood.

Cellulose was removed representing 37.1% of the original wood mass.

The monomer sugars represented 61% of the all sugars in the hydrolyzate as determined by autoclaving the hydrolyzate with 4% H₂SO₄ in 121° C. for 60 minutes, which converted the remaining sugars in their corresponding monomers.

Half of the hydrolyzate was processed without the final reaction step. Calcium oxide was added to reach pH of 11 in the hydrolyzate and the precipitate containing calcium lignosulfonates was filtered off. The cooking ethanol was distilled off until the boiling point of the distillate reached 100.5° C. and density of 0.995 g/mL. The furfural content was determined to be 0.29 g/L in the untreated hydrolyzate after the lignin removal and evaporation step.

The second half of the hydrolyzate was subjected to the final reaction step by injecting 3% by weight of sulfur dioxide and heating for 30 minutes at 140° C. Calcium oxide was added to reach pH of 11 in the hydrolyzate and the precipitate containing calcium lignosulfonates was filtered off. The cooking ethanol was distilled off until the boiling point of the distillate reached 100.5° C. and density of 0.995 g/mL. The furfural content was determined to be 0.06 g/L in the hydrolyzate after the final processing step.

The untreated hydrolyzate, i.e., that was not subjected to the final reaction step, and the treated hydrolyzate, i.e., that was subjected to the final reaction step, were both prepared for fermentation by neutralizing with acetic acid, adding sodium citrate and commercial nutrient broth. Initial sugar composition and subsequent hydrolyzate composition were determined in HPLC.

Fermentation of both hydrolyzates was performed in a laboratory setting using saccharomyces cerevisiae yeast for at least 72 hours at 35° C.

The yield of ethanol from the untreated hydrolyzate corresponded to only 18.6% stoichiometric yield of the original oligomer sugars and monomer sugars present in the hydrolyzate.

TABLE 1
Monomer sugar concentration of the hydrolyzate and the product ethanol concentration
as a function of fermentation time for the untreated hydrolyzate.

| | Glucose | Xylose | Galactose | Arabinose | Mannose | Total Sugars | Ethanol |
|---|---|---|---|---|---|---|---|
| Fermentation | Conc. | Conc. | Conc. | Conc. | Conc. | Conc. | Conc. |
| Time (hours) | (g/L) | (g/L) | (g/L) | (g/L) | (g/L) | (g/L) | (g/L) |
| 0 | 9.33 | 11.83 | 5.30 | 1.94 | 12.05 | 40.45 | 0.00 |
| 24 | 7.55 | 13.91 | 6.17 | 1.69 | 13.22 | 42.54 | 3.76 |
| 48 | 5.85 | 14.79 | 6.71 | 1.84 | 13.48 | 42.67 | 5.57 |
| 72 | 4.41 | 14.74 | 6.68 | 1.74 | 12.72 | 40.29 | 6.30 |

The yield of ethanol from the hydrolyzate treated in the final processing step corresponded to 46.5% stoichiometric yield of the original monomer and oligomer sugars in the hydrolyzate or 2.5 times greater than the amount from the untreated hydrolyzate.

TABLE 2
Monomer sugar concentration of the hydrolyzate and the product ethanol concentration as
a function of fermentation time for the hydrolyzate treated in the final reaction step.

| | Glucose | Xylose | Galactose | Arabinose | Mannose | Total Sugars | Ethanol |
|---|---|---|---|---|---|---|---|
| Fermentation | Conc. | Conc. | Conc. | Conc. | Conc. | Conc. | Conc. |
| Time (hours) | (g/L) | (g/L) | (g/L) | (g/L) | (g/L) | (g/L) | (g/L) |
| 0 | 8.85 | 10.34 | 4.63 | 1.77 | 10.99 | 36.58 | 0.00 |
| 24 | 4.31 | 9.23 | 4.13 | 1.19 | 8.81 | 27.67 | 3.53 |
| 48 | 0.99 | 9.79 | 4.47 | 1.22 | 7.24 | 23.71 | 7.05 |
| 72 | 0.00 | 6.76 | 3.22 | 1.89 | 3.05 | 14.22 | 14.21 |

It should be understood that the terms recited herein are to be given their broadest possible meaning and not restricted to their common dictionary meaning.

1. A process for fractionating lignocellulosic material in to chemically reactive components comprising: a staged treatment of the lignocellulosic material with a solution of aliphatic alcohol, water and sulfur dioxide with intermediate separation of cellulose and hemicelluloses-lignin fractions which are then each further treated with a solution of aliphatic alcohol, water and sulfur dioxide or acid.

2. A process according to claim 1 wherein said solution of aliphatic alcohol, water and sulfur dioxide contains 40% to 60% water.

3. A process according to claim 1, wherein a different concentration of said solution of aliphatic alcohol, water and sulfur dioxide is used at a first stage of treatment of said lignocellulosic material than is used in one or more subsequent stages of treatment with intermediate removal and preservation of cellulose.

4. A process according to claim 1, wherein a sulfur dioxide solution of 3% to 20% is used at a first stage of treatment and a sulfur dioxide, sulfurous acid or sulfuric acid solution of 0.5% to 20% is used in one or more subsequent stages of treatment with intermediate removal and preservation of cellulose.

5. A process according to claim 4, wherein said process is followed by steam stripping and/or evaporation of hydrolyzate to remove and recover sulfur dioxide and aliphatic alcohol and to remove fermentation inhibitors.

6. A process according to claim 1, wherein a sulfur dioxide solution of 0.5% to 9% is used at a first stage of treatment and a sulfur dioxide, sulfurous acid or sulfuric acid solution of 0.5% to 20% is used in one or more subsequent stages of treatment with intermediate removal and preservation of cellulose.

7. A process according to claim 6, wherein said process is followed by steam stripping and/or evaporation of the hydrolyzate to remove and recover sulfur dioxide and aliphatic alcohol and to remove fermentation inhibitors.

8. A process according to claim 1, wherein cellulose is further treated with an aqueous solution of aliphatic alcohol in the presence of acid to esterify and render it a reactive chemical feedstock.

9. A process according to claim 8, wherein the esterified cellulose is fermented to aliphatic alcohol.

10. A process according to claim 8, wherein said acid is sulfur dioxide, sulfurous acid or sulfuric acid solution of 0.5% to 20%.

11. A process according to claim 8, wherein preferred conditions are 95% ethanol and 5% sulfuric acid at 120° C.

12. A process according to claim 1, wherein said process is carried out at temperatures between 65° C. and 200° C.

13. A process according to claim 1, wherein said process is carried out at for a period of time between 15 minutes and 720 minutes.

14. A process according to claim 1, wherein preferred conditions are an initial treatment using 47% ethanol, 47% water and 6% sulfur dioxide at 140° C. for 2 hours, and following the intermediate removal and preservation of the cellulose, a further treatment of the hemicelluloses-lignin fraction using 2% ethanol, 95% water and 3% sulfur dioxide at 140° C. for 1 hour.

15. A process for producing fermentable sugars from the hemicelluloses of a lignocellulosic material comprising: a staged treatment of the lignocellulosic material with a solution of aliphatic alcohol, water and sulfur dioxide with intermediate removal of hydrolyzate and cellulose.

16. A process according to claim 15, wherein a different concentration of said solution of aliphatic alcohol, water and sulfur dioxide is used at a first stage of treatment of said lignocellulosic material than is used in one or more subsequent stages of treatment with intermediate removal of hydrolyzate and cellulose.

17. A process according to claim 15, wherein said process is carried out at for a period of time between 15 minutes and 720 minutes.

18. A process according to claim 1, wherein aliphatic alcohol is produced from fermenting and distilling hydrolyzed fermentable sugars produced in said process and is then reused in said process.

19. A process according to claim 1, wherein lignin is sulfonated and rendered soluble in aqueous solutions.

20. A process according to claim 1, wherein the concentration of sulfur dioxide and aliphatic alcohol in the solution and the time of cook is varied to control the yield of hemicelluloses vs. celluloses and vs. fermentable sugars.

21. A process according to claim 1, wherein excess sulfur dioxide is released from said further treatment of each fraction and used for make-up for cooking chemicals.

22. A process for fractionating lignocellulosic material in to chemically reactive components through a staged treatment of the lignocellulosic material with a solution of aliphatic alcohol, water and sulfur dioxide with intermediate separation of cellulose and hemicelluloses-lignin fractions which are then each further treated with a solution of aliphatic alcohol, water and sulfur dioxide or acid comprising the steps of:
a) Cooking under acidic conditions to produce hydrolyzed hemicelluloses, cellulose, reactive lignin and sulfonated lignin; b) Washing to separate lignin and hemicelluloses from cellulose in several stages to recover over 95% of the aliphatic alcohol mixed with the cellulose; c) Diverting the cellulose to papermaking or treatment of cellulose with an aqueous solution of aliphatic alcohol in the presence of acid to esterify the cellulose, rendering it reactive and thereby a suitable chemical feedstock d) Treatment of post washing hydrolyzate with sulfur dioxide and heat to maximize the yield of fermentable sugars and to remove, and/or neutralize fermentation inhibitors; e) Evaporation to remove and recover cooking chemicals, remove side products, precipitate reactive native lignin and concentrate lignosulfonates and/or fermentable sugars product; f) Lignin separation to remove reactive native lignin and reactive lignosulfonates from fermentable sugars; g) Fermentation and distillation to produce and concentrate aliphatic alcohols or organic acids; h) Drying the concentrated aliphatic alcohols or organic acids with anhydrous lime and reusing the resulting hydrated lime byproduct for lignin separation; and
i) Fractionation and/or separation to remove and recover side products.

23. A process according to claim 20, further comprising the step of fractionation and/or separation to remove and recover side products.

24. A process according to claim 20, further comprising the step of lignin and/or lignosulfonate separation.

25. A process according to claim 24, wherein aliphatic alcohol soluble lignin is separated by evaporation of said aliphatic alcohol and subsequent removal of reactive native lignin precipitate.

26. A process according to claim 24, where reactive lignosulfonates are selectively precipitated using excess lime in the presence of aliphatic alcohol.

27. A process according to claim 26, where lignosulfonates filter cake is combusted in a fluidized bed boiler or gasifier, sulfur is released and reacts with excess lime in said filter cake to form gypsum.

28. A process according to claim 20, further comprising the step of fermentation and distillation.

29. A process for drying product aliphatic alcohol from distillation comprising using anhydrous lime to remove water from product aliphatic alcohol and reusing resultant hydrated lime byproduct to displace fresh lime in an associated upstream feedstock preparation process.

30. A process according to claim 29, wherein said hydrated lime byproduct is used in claim 26 to precipitate lignosulfonates.

31. A system for removing aliphatic alcohol from a stream and concentrating the resulting stream comprising an integrated alcohol stripper and evaporator system, wherein aliphatic alcohol is removed by vapor stripping, the resulting stripper product stream is concentrated by evaporating water from the stream, evaporated vapor is compressed using vapor compression and is reused to provide thermal energy for both the stripper and the evaporator.

32. A process according to claim 31, wherein evaporated vapor streams are segregated so as to have different concentrations of organic compounds in different streams.

33. A process according claim 31, wherein evaporator condensate streams are segregated so as to have different concentrations of organic compounds in different streams.

34. A process for minimizing effluent discharges from distillation columns and evaporators, wherein distillation column bottoms solution and evaporator condensates are reused to wash cellulose.
